(19) United States
(12) Patent Application Publication
(10) Pub. No.: US 2007/0161820 A1
(43) Pub. Date: Jul. 12, 2007

(54) Simultaneous recovery and continuous extraction of substantially pure carboxylic acids and ammonium salts from acid hydrolysis reaction mixtures

(76) Inventors: David J. Sanders, Clive, IA (US); Tommy Vinh Truong, Urbandale, IA (US); Vincent Sewalt, West Des Moines, IA (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/330,891

(22) Filed: Jan. 12, 2006

(51) Int. Cl. C07C 51/08

(52) U.S. Cl. 562/606

(57) ABSTRACT

A continuous or batch process for the production and recovery of substantially pure carboxylic acids and ammonium salts from a corresponding nitrile. By controlling the amount of water used in the reaction, a biphasic reaction mixture forms which facilitate simultaneous recovery of both fractions. In a preferred embodiment, using propionitrile as the starting material and strong sulfuiric acid, propionic acid of approximately 98% purity is produced in the upper phase. A combination of hydrochloric and sulfuric acid can reduce the total reaction volume.

### BACKGROUND OF THE INVENTION

The invention relates generally to the conversion of nitrites into carboxylic acids using acid hydrolysis and, more specifically, to the substantially simultaneous recovery and continuous extraction of substantially pure carboxylic acids and ammonium salts from acid hydrolysis reaction mixtures.

Propionic acid is an organic acid that has wide use as a mold inhibitor for grains, forage, silage, and animal feeds and human food. Propionic acid is a generally recognized safe (GRAS) substance as a chemical preservative when used in accordance with applicable regulations and good manufacturing and feeding practices. Propionic acid is produced biologically from the metabolic breakdown of fatty acids containing odd numbers of carbon atoms, and also it is a breakdown product of some amino acids. Bacteria of the species Propionibacteria produce propionic acid as the end product of their anaerobic metabolism. Large amounts of propionic acid were once produced as a by product of acetic acid manufacture using cobalt as a catalyst, but development of the rhodium- and indium-catalyzed reaction with reduced levels of by-product have made this a very minor source of propionic acid today.

On an industrial scale, propionic acid today is usually made by one of three process routes: Direct oxidation of hydrocarbons; ethylene hydroformulation/oxidation; and carboxylation of ethylene with carbon monoxide and water. Currently, the world's largest producer is BASF with approximately 176 million pounds per year production capacity. BASF uses the direct carboxylation of ethylene process. Because this process is directly dependent on ethylene, which is petroleum-derived, the price of propionic acid is heavily dependent on petroleum prices.

Propionitrile is a by-product from the manufacture of chemical intermediates in the production of nylon and is an attractive starting material in the production of propionic acid. Several methods are available for the hydrolysis of nitrites to corresponding organic acids. Acid and base hydrolysis are the classical methods used. In base-catalyzed reactions of propionitrile, ammonia and the corresponding metal propionate are produced. For example, a calcium hydroxide reaction would give calcium propionate along with ammonia gas. Acid-catalyzed reactions with nitrites yield free propionic acid and the ammonia salt of the mineral acid. Accordingly, for example, hydrochloric acid hydrolysis of propionitrile would give propionic acid and ammonium chloride. Others have evaluated the use of base-catalyzed hydrolysis of propionitrile. These reactions are chemically feasible, especially with calcium oxide/hydroxide, and are the subject of one or more patents. Although the resulting product enjoys GRAS approval it has been deemed not commercially viable.

### SUMMARY OF THE INVENTION

The invention consists generally of the conversion of a nitrile to the corresponding carboxylic acid using a strong acid, or combination of strong acids and water. In one embodiment, the invention consists of a propionitrile-to-propionic acid conversion process via sulfuric acid hydrolysis, yielding ammonium bisulfate in addition to propionic acid. Reaction conditions have been refined to optimize the reaction rate and yield of propionic acid. Based on the examples, the important reaction conditions include using near stoichometric reaction amounts of propionitrile, with slight excess of water (preferably between about 3 and 4 moles), and sulfuiric acid heated to at or above 85-90° C. using the heat of hydration of sulfuric acid, with formation of two separate and substantially pure reaction phases. Propionitrile hydrolysis is near complete after 5-7 h with the hydrolysis of the intermediate product, propionamide, to propionic acid, taking place essentially concurrently. Propionic acid in the upper phase is greater than 98% pure and can be used for formulation into mold inhibitor products. When properly controlled, there will be less than 15% by volume of propionic acid in the lower phase. Ammoniumsulfate, produced from the generated ammonium bisulfate by reaction with ammonia, can be sold as a source of nitrogen fertilizer, or, if sufficiently pure, as reagent to purify proteins. The control of the water content in the reaction is of paramount importance, as it allows bi-phasic separation of substantially pure molecular fractions for easy recovery: As the reaction proceeds, the water content drops to a point where propionic acid is forced into a single, substantially concentrated phase, allowing it to be easily recovered, and the lower phase gains increasing concentrations of ammonium bisulfate, which can be separated for use. The mole ratio of water:sulfuric acid is at least 2.5 and water:propionitrile is at most 6 and preferably between about 3 and 4.

In another embodiment, hydrochloric acid is used in combination with sulfuric acid. The addition of a controlled amount of hydrochloric acid can also decrease the total reaction volume while maintaining both the biphasic nature of the reaction and high purity of propionic acid in the upper phase as well as leaving only trace amounts of unreacted propionitrile in the two layers.

In an alternative embodiment, the reaction is carried out continuously in two or more reaction vessels. The starting materials are continuously added and combined in the first reaction vessel, heated, and allowed to react, preferably under stirring. When the average residence time in the first reaction vessel is sufficient so that the reaction has proceeded to preferably greater than 50% completion, a portion of the reaction mixture is transferred to a second reaction vessel. The flow of starting ingredients into the first reaction vessel and outflow of reaction mixture to the second reaction vessel are adjusted to be substantially equal over time. The reaction mixture in the second reaction vessel is also heated but not stirred so that the two phases develop. The reaction mixture in the second reaction vessel is allowed to react substantially to completion. The two phases are drawn off separately from the second reaction vessel at a rate that is substantially equal over time with the in-flow of the reaction mixture from the first reaction vessel.

Although the examples shown here are all reactions at atmospheric pressure, anyone skilled in the art will be able to duplicate the experiments at elevated pressures and concomitant temperatures above those included here.

### BRIEF DESCRIPTION OF THE FIGURES

FIGS. 1A-D are chromatographs of samples taken at 2 and 3.3 hours; A—2 Hr sample at full scale, B—3.3 Hr sample at full scale, C—Overlaid and zoomed in chromatograph of 2 and 3.3 Hr samples, D—Enlargement of the PN peak area (Rx 8).

FIG. 2 is a chart of reactant, intermediate and final product concentrations through the first 3.3 hours of reaction (Rx 9).

FIG. 3 is a chart of reactant, intermediate and final product concentrations for up to 29 hours (Rx 10).

FIGS. 4A-B are vial 5 chromatographs of upper layer samples taken at 2 and 3 h with standard propionic acid on (A) full scale and (B) expanded scale (Rx 9).

FIGS. 5A-D are gas chromatograph profiles showing PN and PA in the different distillation fractions; the first peak is PN and the second peak is PA; A—Initial upper phase, B—Upper phase after distillation, C—Upper phase of distillate, D—Lower phase of distillate.

FIGS. 6A-C are charts of the surface response analysis into the impact of H₂SO₄:PN and water:PN on reaction rate and yield in Experiment 1 after (A) 3h, (B) 7.5 h, and (C) 13 h; yield is expressed as the moles of ABS generated as a percentage of the initial concentration of PN present in the reaction; the optimum reactant ratios as determined in the surface analysis are 1.22 moles sulfuric and 4.02 moles of water with a reaction time of 11.6 h.

FIGS. 7A-B are charts of the experiment 2 surface response of PA yield (%) to varying water:PN ratio after reaction for the indicated reaction times; (A) upper phase recovery of PA, and (B) PA recovered from the lower phase.

FIG. 8 is a chart showing an overlay of the FTIR spectra of a standard sample of propionic acid and propionic acid produced using the present invention.

### DETAILED DESCRIPTION OF PREFERRED EMBODIMENTS

Carboxylic acids include organic acids that have a carboxyl group, having the general formula RCOOH, wherein the R is a hydrogen or an organic group. Carboxylic acids of applicability to this invention include acetic acid, formic acid, propionic acid, benzoic acid, butyric acid, acrylic acid, lactic acid, oxalic acid, malonic acid, succinic acid, glutaric acid, adipic acid, and salicylic acid.

Strong mineral acids applicable to this invention include but are not limited to hydrochloric acid, nitric acid, and sulfuric acid.

Nitriles are any of a class of organic compounds containing the cyano radical —C ≡N. Nitriles applicable to this invention include acetonitrile, acrylonitrile, propionitrile, formionitrile, benzonitrile, butyronitrile, lactonitrile, oxalonitrile, malononitrile, succinonitrile, glutaronitrile adiponitrile, and salicylonitrile.

### EXAMPLE 1

### Hydrolosis of Propionitrile to Propionic Acid using Sulfuric Acid

Several strong acids have the potential to be considered for commercial use. Phosphoric, hydrochloric, nitric and sulfuric acids are readily available and should react with propionitrile (PN) to generated propionic acid (PA) and an ammonium salt. From cost estimates, it is apparent that the sulfuric acid reaction is cost-competitive with nitric acid and hydrochloric acid. In addition, it has several other advantages compared to other acids. Sulfuric can be obtained at higher practical maximum concentration (95%) than hydrochloric (36%) and nitric (65%). This is important in controlling the quantity of water remaining at the end of the reaction. If the majority of water is used up in the reaction, the salts can be more easily precipitated and conveniently recovered. A second consideration in selecting an acid is its ability to form the two-phase reaction mixture. A third important factor in selecting an acid is the type of salt formed, and the market potential or disposition cost involved. The quantity of ammonium salt will be substantial, ranging from 42% of the reaction mass (ammonium chloride) to 61% (ammonium (bi)sulfate). One outlet for this large volume of by product salt is as a nitrogen source in agricultural fertilizers. Ammonium sulfate, ammonium nitrate and ammonium phosphate are routinely used in this manner, however ammonium chloride is not. Finally, ammonium nitrate would likely have explosive properties when combined with flammable organic solvents such as propionitrile. With higher cost for phosphoric acid, testing of acid hydrolysis of PN has focused on sulfuric acid.

Sulfuric acid will react with PN to form propionic acid via propionamide according to the following reaction:
CH₃CH₂CN+H₂SO₄+2 H₂O→CH₃CH₂COOH+NH₄HSO₄
With the following reaction steps:
CH₃CH₂CN+H₂SO₄+2 H₂O→CH₃CH₂CONH₂+HSO₄⁻+H₃O⁺CH₃CH₂CONH₂+HSO₄⁻+H₃O⁺→CH₃CH₂COOH+NH₄HSO₄
The salt of this reaction is ammonium hydrogen sulfate, or ammonium bisulfate (ABS). This salt is formed due to the incomplete ionization the second proton in sulfuric acid. There are some indications that some ammonium sulfate can be formed. Ammonium bisulfate is not the preferred salt for use in fertilizers. This salt can however be readily converted to ammonium sulfate by the addition of ammonium ions to solutions of ammonium bisulfate (U.S. Pat. No. 4,950,788) as follows:
NH₄HSO₄+NH₃→(NH₄)₂SO₄

The following table includes the molar ratios to convert propionitrile into propionic acid and the production of ammonium sulfate from the generated ammonium bisulfate.

TABLE 1
Reaction mass balance for sulfuric acid catalyzed
conversion of propionitrile into propionic acid.

| | Molar | Molecular | Wt. % |
|---|---|---|---|
| | Ratio | Wt. | of Rx |
| Reactants | | | |
| PN | 1 | 55.08 | 26.7% |
| H₂SO₄ | 1 | 98.08 | 47.6% |
| H₂O | 2 | 18.016 | 17.5% |
| NH₃ | 1 | 17.03 | 8.3% |
| Products | | | |
| PA | 1 | 74.08 | 35.9% |
| (NH₄)₂SO₄ | 1 | 132.14 | 64.1% |

In order to determine whether these assumptions are reasonable, a series of experimental hydrolysis reactions were conducted. Small lab scale reactions were run to determine the best practical reaction conditions in order to utilize this reaction.

### Materials and Methods

A 100-mL three-neck round bottom flask was set up with a water-cooled reflux condenser, an addition funnel and a thermometer. With magnetic stirring, propionitrile (Sigma-Aldrich 99%) and DI water were added into the flask and concentrated sulfuric acid (Fisher Scientific 95.9%) was added slowly into this mixture using the addition funnel. A first exploratory reaction was run was at approx. 70° C. for 24 hours, during which time small aliquots of reaction mix were taken for ammonia determination spectrophotometrically using Nessler's reagent and PN and PA determination by gas chromatography. The vessel was allowed to cool to room temperature and the contents stored at room temperature for several weeks, after which components were separated, weighed and tested by GC analysis. In subsequent reactions, the temperature was held between 85° and 92° C. and the reaction was allowed to cool to room temperature overnight followed by separation of the reaction phases, which were then weighed and assayed by GC for PN and PA content. Reactions 9-11 were set up with a molar ratio optimized for the reaction stoichiometry yielding ammonium bisulfate rather than ammonium sulfate. Initial reactant mole ratios for Rx 2-11 are indicated in Table 2.

TABLE 2
Reactant mole ratios for test reactions 1-8

| | Reaction | Reactant | | |
|---|---|---|---|---|
| Test | Time | Mole Ratio | | |
| Run | (Hr) | PN | H₂SO₄ | H₂O |
| Rx 2 | 6 | 2.4 | 1.0 | 2.0 |
| Rx 3 | 6 | 1.7 | 1.0 | 3.4 |
| Rx 5 | 19 | 1.5 | 1.0 | 4.0 |
| Rx 6 | 19 | 1.3 | 1.0 | 4.0 |
| Rx 7 | 19 | 1.6 | 1.0 | 3.4 |
| Rx 8 | 3.3 | 1.0 | 1.2 | 2.1 |
| Rx 9 | Up to 48 | 1.0 | 1.0 | 2.0 |
| Rx 10 | Up to 30 | 1.0 | 1.0 | 3.1 |
| Rx 11 | 13 | 1.0 | 1.0 | 2.5 |

The time course reactions, Rx 9 and 10, were run using a series of 40 mL glass vials fitted with 8″(⅛″ID) glass tube condensers. These tube condensers prevented PN vapors from escaping and allowed the reactions to be run at atmospheric pressure. For each experiment twelve vials were set up to contain reactants and the vials were placed into a 90° C. shaking water bath. Rx 9 and 10 differed from each other only in the amount of water in the initial reaction mix, see Table 2. Over time single vials were removed from heat and diluted with 25 ml of de-ionized water. The amount of dilution water was sufficient to allow single-phase solutions to be assayed, i.e. the final solution concentration contained <10% PN.

The densities of phases were measured using a 100 μL Hamilton gas-tight syringe at room temperature. One hundred (100) μL of the collected phase was drawn into syringe and weighed to the nearest 0.1 mg on a Mettler AE160 balance. GC analysis of reaction phases was conducted using the syringe volume from the density measure with the weighed syringe contents transferred into sealed GC vials containing 1 mL of 1 M HCl and 0.4 mL of 2 N NaCl. Vials were vortexed and injected onto a GC with the conditions shown in Table 3. A stock standard solution was prepared with 50:50 wt.:wt. propionitrile (99%) and propionic acid (99%) obtained from Sigma-Aldrich and Fisher Scientific, respectively. Working standard solutions of PN and PA were prepared to cover the entire range of concentrations listed in Table 4. The working standards were prepared for analysis in the same manner as the samples.

TABLE 3
Gas chromatography conditions for analysis of propionitrile and
propionic acid

| | |
|---|---|
| Column: | 15 m Nukol 0.25 id, 0.25 um film |
| Instrument: | Perkin Elmer Autosystem |
| Oven: | 80° C. for 0.5 min. ramped at |
| | 20° C./min. to 200° C. |
| Carrier: | Helium at 18 psi |
| Injection: | split inj. 0.5 uL w/ 300:1 split ratio |
| Injector: | 180° C. |
| Detector: | 220° C. |
| Data System: | Turbochrom Workstation |

Ammonia determination was conducted using Nessler's reagent. Aliquots (1-2 mL) of test solutions were transferred into 4 mL de-ionized water using a Hamilton 10 uL syringe. 100 uL of Nessler's Reagent (Aqua Solutions, CY183A) was pipetted into test tubes and vortexed at 1400 rpm for 3-4 seconds. Standard solutions of ammonium sulfate were prepared in the same manner and the color was allowed to develop for 20-30 minutes. Tubes were read on a Beckman DU640 spectrometer at 340 nm.

### Results

In general, the reactions started biphasic, however within 15 minutes the solution was cloudy with a much smaller upper phase. As the reaction progressed the upper phase became larger to a maximum after several hours. Temperatures rose initially with the addition of sulfuric acid and the addition rate was limited to keep pot temperature from rising too rapidly. Maximum temperatures after addition of acid were 60-70° C.

The results of GC analysis of the separate phases from the test reactions are shown in Table 4. The molar ratios of reactants are given in Table 5 along with the recovery based on the limiting reagent, which in all cases was sulfuric acid. Calculation of the PN recovery was based on the concentration in the reaction minus the assayed levels from both phases divided by the equivalent moles of limiting reagent (sulfuric acid). PA recovery was calculated by dividing the assayed PA from both phases by the equivalent moles of limiting reagent.

TABLE 4
PA and PN analysis results from organic (upper) and
aqueous (lower) phase from test reactions.

| | Reaction | Upper Phase | | | | Lower Phase | | | |
|---|---|---|---|---|---|---|---|---|---|
| Test | Time | Measured | Density | | | Measured | Density | | |
| Run | (Hr) | (Wt. %) | (g/ml) | PN % | PA % | (Wt. %) | (g/ml) | PN % | PA % |
| Rx 2 | 6 | 47.2 | 0.870 | 51.6 | 46.3 | 52.8 | 1.45 | 0.9 | 1.6 |
| Rx 3 | 6 | 49.3 | 0.936 | 36.0 | 56.6 | 50.7 | 1.50 | 0.6 | 2.0 |
| Rx 5 | 19 | 42.3 | 0.945 | 27.2 | 62.5 | 57.7 | 1.50 | 0.4 | 2.3 |
| Rx 6 | 19 | 36.7 | 0.967 | 21.3 | 69.4 | 63.3 | 1.52 | 0.9 | 2.0 |
| Rx 11 | 13 | 34.05 | | 0.7 | 98.0* | 63.51 | | 0.3 | 2.2 |

TABLE 5
Reactant mole ratios and molar recovery of PN and PA based
GC analysis of the limiting reagent

| | Reaction | Reactant | | | Molar Recovery, % | |
|---|---|---|---|---|---|---|
| Test | Time | Mole Ratio | | | PN | PA |
| Run | (Hr) | PN | H₂SO₄ | H₂O | Recovered | Recovered |
| Rx 2 | 6 | 2.4 | 1.0 | 2.0 | 105.3 | 88.2 |
| Rx 3 | 6 | 1.7 | 1.0 | 3.4 | 93.5 | 89.4 |
| Rx 6 | 19 | 1.3 | 1.0 | 4.0 | 93.6 | 86.7 |
| Rx 11 | 13 | 1.0 | 1.0 | 2.5 | 98.5 | 92.8 |

The results for Rx 2-6 show levels of PN remaining and the PA generated ranging from 21.3% to 51.6% PN in the upper phase and 46.3% to 69.4% for PA concentrations. The lower phase has much higher consistency with levels of PN below 1% and PA levels less than 3%. Molar recovery values are nearly equivalent for this series of reactions given the potential for error in accurately measuring phase weights and analytical error in the GC method. This was true even after only 6 hours of reaction, which would seem to argue that the reaction might be fairly complete in that time.

In order to achieve low levels of PN in the final product, the reaction must go to near completion or the PN must be easily removed, e.g., by distillation. In Rx 1 to 7 the reactant ratios were optimized for the assumed stoichiometry involved in generating ammonium sulfate as the primary salt rather than ammonium bisulfate. Additional reactions with PN being the limiting reagent were run to determine the degree of completion to which the reaction could be driven.

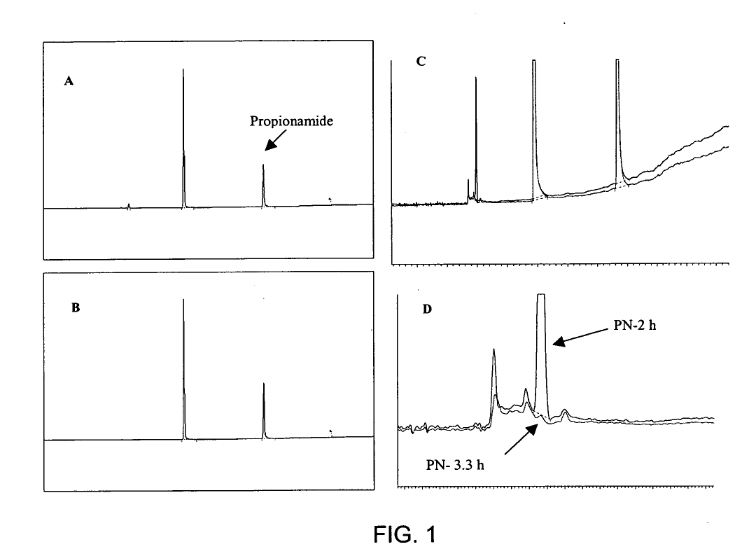

Rx 8 was run with molar ratios of 1:1.2:2.1 of PN: sulfuric acid:water and the total reaction time was 3.3 hours. After reaction the mixture was a single-phase and did not separate after cooling, as did the previous reactions. The chromatographs in FIG. 1 show that only traces of the PN peak are present after 3.3 hours and show why propionic acid recovery after 3.3 hours was only 61.3%. At 3.3 hours nearly all PN has reacted but the second step in the conversion, the hydrolysis of propionamide to propionic acid is incomplete. Because this hydrolysis is not complete there would still be considerable water in the reaction vessel which will solvate propionic acid and prevent the formation of an organic phase.

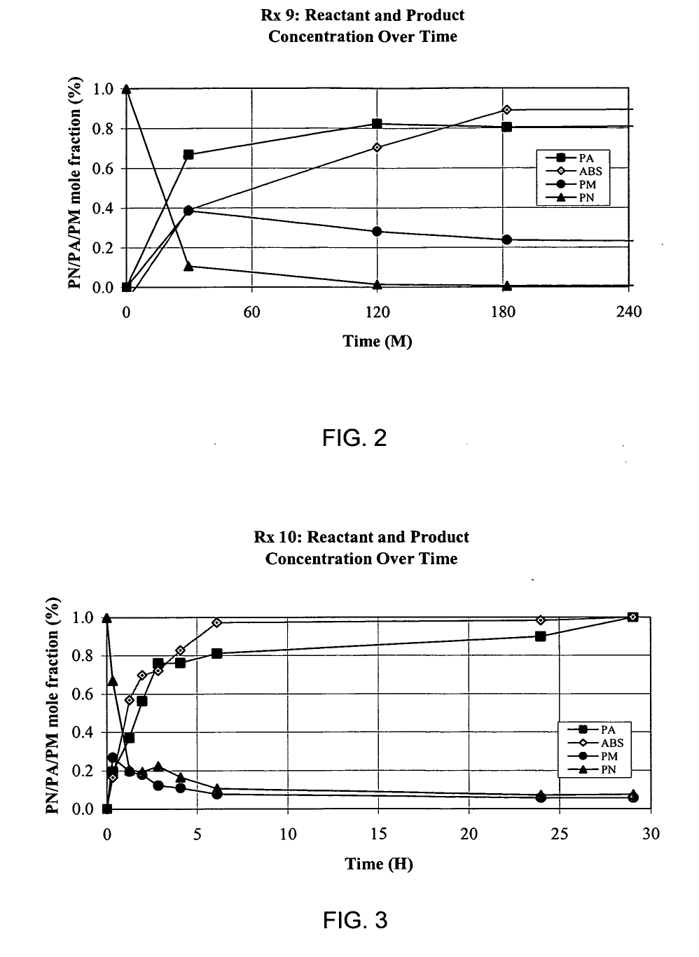

The concentration of total ammonium ions released from the nitrile group during hydrolysis is a direct measure of the conversion of propionitrile into propionic acid. More specifically, during the reaction ammonium bisulfate is formed after hydrolysis of propionamide, the intermediate product of propionitrile hydrolysis. FIG. 2 shows the results of the time series reaction. The results shown are the results of GC analysis for PN, PA, and PM and ammonia determination by Nessler's reagent to estimate ammonium bisulfate levels. The concentrations are in millimolar ratios, a method that allows a simple comparison of initial reactant moles to product moles. Based on the actual concentration of PN added to the vials of 73 mmoles, we should expect the same number of moles of PA and ABS to be generated. The average concentrations for samples run for nearly 2 days were <0.1 mM PN, 77.1 mM PA and 2.2 mM PM. ABS concentration was estimated at 57.9 mM, which is lower than the expected 73 mM.

FIG. 3 graphs the results of the second time course reaction, Rx 10. The scale is normalized to the maximum mole quantity analyzed. From the data it does appear that the reaction progresses rapidly for the first 5 h and is essentially complete after 10 h. Propionic acid results in this assay are lower than would be expected at 13 h in comparison to the results seen in Rx 11 possibly due to analysis variation or inadequate mixing in the vials.

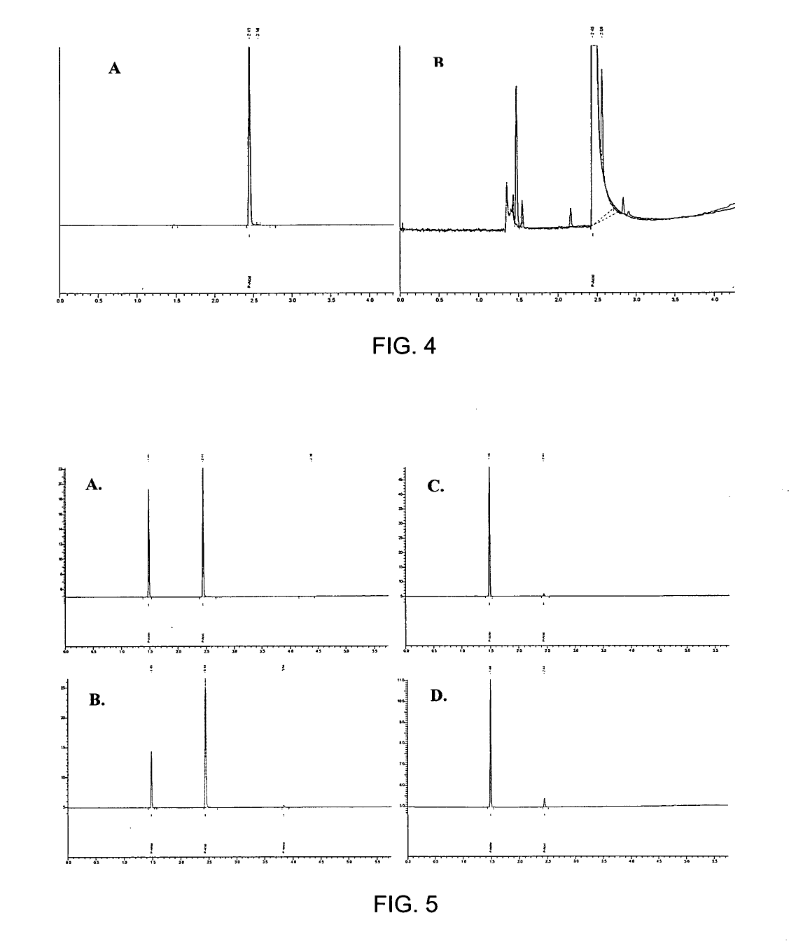

FIG. 4 shows gas chromatographic results of a test vial taken from this test. Vial number 5 had an additional 1.5 g of water added at about 3 h into the reaction. This allowed the sample to remain biphasic and prevented crystallization of ABS at 90° C. After two days at 90° C. the sample was allowed to cool which forced the crystallization of the lower phase. The upper phase was decanted and the weight was measured. This phase was assayed for PN, PA and PM as well as ammonium bisulfate concentration. The results of upper phase analysis showed nearly pure propionic acid, 0.25% residual PN, no detected PM and ammonium bisulfate was below detection, confirming a complete separation of organic and aqueous phases. FIG. 4 shows an overlay chromatograph of Rx 9 Vial 5 upper phase compared to an analytical grade sample of propionic acid.

Upon cooling of the reaction flask from Rx 3 to room temperature, the ammonium bisulfate crystallized into a solid in the lower phase. The lower phase is highly saturated with ammonium bisulphate and will readily crystallize. Levels of PN and PA in the lower phase were below 1% and 3%, respectively, with the amount of PA pulled into the lower phase seemly dependent on concentration of residual water in the lower phase.

In order for this process to be most useful, the final salt should be in the form of ammonium sulfate. Addition of ammonium hydroxide to the lower phase was conducted on a 10 g sub-sample of the lower phase from Rx 7 in order to determine whether ammonium sulfate could be generated. The sample was stirred and pH was monitored as ammonium hydroxide was injected below the surface. Some heat was generated during addition and the final pH ended up as 8.2. A total of 9.17 g of 29% ammonium hydroxide was added. An excess of ammonia was added as is evident by the above neutral pH value and some vapors were lost during the experiment. The solution crystallized readily with minimal cooling. Crystals were collected from this experiment and from the un-neutralized lower phase. Both were dried at 95° C. in a vacuum oven for 2 h. The melting points were approximately 105° C. for the un-neutralized and >280° C. for the neutralized crystals. The published melting points are 147° C. for ammonium bisulfate and a decomposition point (melting point) of 280° C. for ammonium sulfate. In another experiment, anhydrous ammonia was bubbled through a sample of the lower reaction phase. As ammonia was bubbled through the solution white precipitate formed immediately and after sufficient addition the entire solution solidified.

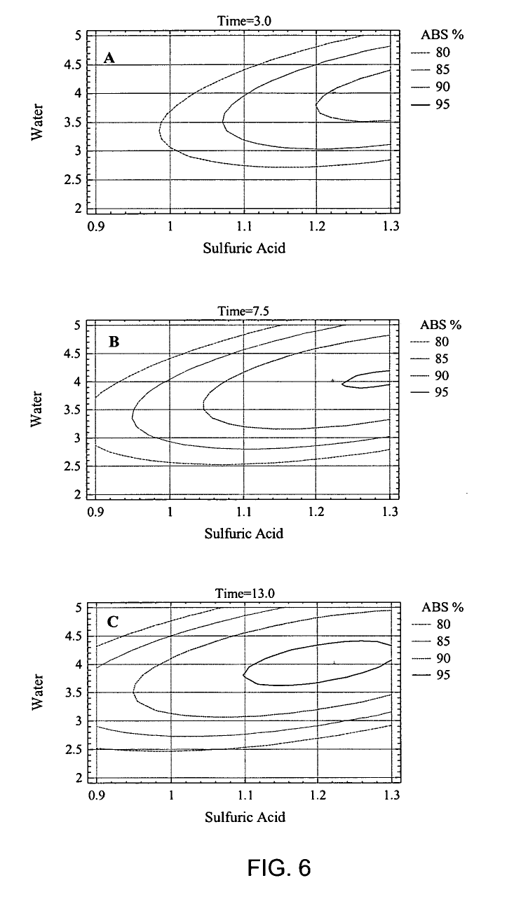

To investigate the ease of separation of residual PN from the reaction a simple distillation was run on the upper phase from Rx 7. The reaction flask was fitted with a small water-cooled condenser attached to a small receiving flask. Heat was applied using an oil bath set to 210° C. and temperatures were monitored in the flask bottoms and at the still head. Distillation was run until the bottoms temperature was 130° C. at which time the heat was shutdown and the flask was allowed to cool. A total of 5.6 g of distillate was collected of which 0.7 g was a separate heavier phase. The fraction were tested for density and GC levels and the results are included in Table 6 and the GC profiles are included in FIG. 6.

TABLE 6
Summary of distillation experiment

| | | | | [PN], | [PA], |
|---|---|---|---|---|---|
| | | | Fraction | wt % | wt % |
| ID | Fraction | Density | Wt. | Solution | Solution |
| A | Upper phase initial | 0.917 | 34.5 | 28.7 | 74.3 |
| B | Upper phase after | 0.967 | 27.5 | 16.2 | 89.7 |
| | distillation | | | | |
| C | Upper phase distillate | 0.798 | 4.9 | 90.5 | 4.5 |
| D | Lower phase distillate | 0.981 | 0.7 | 10.5 | 1.9 |

As can be seen in the results of distillation of the upper phase, a reduction in residual PN is accomplished. The initial PN levels were reduced by 12.5% and PA was concentrated to near 90%. More importantly, distillation of PN seems to be a simple distillation with no azeotrope forming with propionic acid. The boiling points for PN and PA are 97.2° C. and 141° C., which should allow good separation of these components. Additionally, a small amount of water was distilled and recovered as is apparent in the collected lower phase distillate. With reduced water content, residual ammonium bisulfate was crystallized as the bottoms cooled, which leads to a more pure final solution.

### Discussion

In general, the reaction of sulfuric acid and propionitrile appears proceed satisfactorily. An important consideration is the control of the water content. As the reaction proceeds, water is consumed by addition to propionitrile. The upper phase remains an organic layer and the lower gains increasing concentrations of ammonium bisulfate. As the water content of the lower phase decreases, PA is driven into the organic phase with PN. This reduction in water also causes the ammonium salts to approach their solubility limit. This can be very helpful in collecting the salts as crystals with little energy input as would be necessary with evaporation to force crystallization. Several additional methods for control of the water content could be employed. Distillation of reaction contents could not only remove residual PN from the final product, but would help remove residual ammonium sulfates.

An alternative to distillation to remove water could be to contact the final reaction phases with fresh concentrated sulfuric acid. This acid would draw up water from the lower and upper phases and then this would be recycled into subsequent reaction batches.

Addition of ammonia into the lower phase could have the following advantages: formation of ammonium sulfate from the bisulfate, helping to control crystallization by reducing solubility in water compared to bisulfate and neutralizing any excess sulfuric acid from the reaction.

The control of the balance water content in the reaction is of paramount importance, as it allows bi-phasic separation of substantially pure molecular fractions for easy recovery: (1) As the reaction proceeds, water is consumed by addition to propionitrile, and the molar ratios of water to PN is kept as close to 2:1 as possible, which as the reaction proceeds, drops the water content to a point where propionic acid is forced into a single, substantially. concentrated phase; (2) with very limited water, the upper phase remains organic, allowing it to be easily recovered; and (3) the lower phase gains increasing concentrations of ammonium bisulfate which can be precipitated. The reduction in water also causes the ammonium salts to approach their solubility limit.

### EXAMPLE 2

### Acid Hydrolosis of Propionitrile to Propionic Acid using Hydrochloric Acid Alone or Combined with Sulfuric Acid

Propionitrile (PN) can be hydrolyzed with sulfuric acid (H₂SO₄) in a biphasic reaction, allowing for the recovery of substantially pure propionic acid (PA) and concentrated ammonium bisulfate (NH₄HSO₄). Experiments were conducted to determine whether other strong acids could also be employed to convert PN to PA in a manner similar to H₂SO₄ hydrolysis. In addition to H₂SO₄, other readily available strong acids include hydrochloric (HCl) and nitric acid (HNO₃).

This example focuses on HCl (pKₐ, −8) as an alternative to H₂SO₄ (pKₐ₁, −3; pKₐ₂, 2.0). The experimental results show that HCl rapidly catalyzes PN hydrolysis. In addition, HCl has the advantage of producing NH₄Cl as the co-product, which can be utilized in a variety of applications without further reaction, unlike NH₄HSO₄ resulting from H₂SO₄ hydrolysis of PN. Furthermore, HCl is unlikely to undergo an interfering oxidation-reduction reaction, contrary to nitric acid (HNO₃). It is one of the least hazardous strong acids to handle; despite its acidity, it produces the less reactive and non-toxic chloride ion. Finally, HCl is already safely used in many chemical reactions to produce food ingredients or feed additives, including aspartame, fructose, citric acid, lysine, hydrolyzed protein, and gelatin.

Hydrochloric acid is produced in solutions up to 38% HCl (concentrated grade). Bulk industrial grade is between 30 and 34%, optimized for effective transport and limited product loss by HCl vapors.

Three processes utilizing HCl were explored. Process I was the hydrolysis of PN with concentrated HCl acid (37.2%) at a constant high temperature (95° C.). Process II was the hydrolysis of PN with concentrated HCl acid (37.2%) initially reacted at 55° C. and then heated to 95° C. Process III was the hydrolysis of PN with mixtures of HCl (37.2%) and concentrated H₂SO₄ at a constant temperature.

### Materials and Methods

Reaction apparatus. All reactions were run using a 250 mL round flat bottom flask fitted with glass condenser and thermometer, except reaction in the bomb vessel. Heating was provided from an oil/water bath (mineral oil added onto water) and a heat block with stirring option. Reaction progress was monitored by the appearance of white precipitate ammonium chloride crystal. Product separation was performed using vacuum filter with Baxter S/P filter paper (Cat. F2330-90). A condenser was used in every reaction, except in the bomb vessel.

Analytical. The following analytical methods were used to analyzed the collected products: PA by gas chromatography as described below; percent volatiles weight loss, percent water by Karl Fisher Water Titration, residual PN by gas chromatography as described below, and molecular characterization of PA by FT-IR.

The gas chromatography conditions for trace analysis of PN was as follows:

| | |
|---|---|
| Column: | Supelco 24017(0.25 mm ID × 30 m, 0.25 Film μm) |
| Instrument: | Perkin Elmer Autosystem |
| Oven: | 90° C., hold for 0 min |
| | Ramp: 20.0° C./min to 140° C., hold for 4.00 min |
| Carrier: | Helium at 22 psi |
| Injection: | Split flow ratio: 30.0 mL/min. Injection volume: 1 μL |
| Injector: | 180° C. |
| Detector: | 250° C. |
| Data System: | Turbochrom Workstation |

Processes. Reaction Process I involves introduction of PN, HCl and H₂O into the reactor in ratios ranging from 1:1:3.4 to 1:1.7:5.8 (see Table 7), which is then immediately heated to 95° C. in the water bath. Agitation was supplied from a magnetic stir bar throughout the reaction. The products were cooled to room temperature before performing vacuum filtration, either with an ice bath or cold water bath. The collected products were analyzed using the above listed methods. The results are presented in Table 7.

Process II is similar, but after the reactants were added into the reactor flask, they were initially reacted at a water bath temperature of 55-60° C. When the reactor reached a steady temperature, supplemental heat was supplied to achieve 95° C. for the remainder of the experiment. Agitation was supplied from a magnetic stir bar throughout the reaction time. Similarly to Process I, products were collected and analyzed. The results are presented in Table 8.

Process III involves simultaneous reaction with both HCl and H₂SO₄ in combination. Hydrochloric acid was first introduced in the reactor flask and H₂SO₄ was added slowly into the flask. After adding the acids, PN was transferred into the flask without supplying heat, immediately. When the reactor's temperature reached a steady temperature, it was placed in the heated water bath (95 to 98° C.) for the rest of the experiment. As in the previous processes, products were collected by vacuum filtration. Table 9 is the list of experiments that were run in Process III. Experiment 7 is a replication of experiment 1 and 2 for the determination of reaction efficiency. The collected product in this reaction was diluted in water and total PA concentration was determined. A calculation was performed from the theoretical PA versus the collected PA yields the reaction percent conversion. The collected PA upper phase was characterized by FT-IR and compared to a pure PA sample.

### Results

Process I. Within 20 min of reaction time, all experiments in Process I have shown the progress of the hydrolysis reaction with the formation of white precipitate NH₄Cl. When cooled to room temperature, the finished product contained a liquid solution and NH₄Cl precipitate. By vacuum filtration, the precipitate was separated from the liquid. Analytical tests were conducted to determine the composition of each phase. Table 9 is a summary of the results from all experiments that had been conducted under these process conditions.

TABLE 7
Reactant mole ratios and result summary for reaction runs - Process 1¹.

| | Reactant Mole Ratio | | | | Liquid Phase | | | | Wet Precipitate (NH₄Cl) | | | |
|---|---|---|---|---|---|---|---|---|---|---|---|---|
| | PN | | | Added | Mass | PA | H₂O | PN | Mass | PA | H₂O | PN |
| Reaction | (mass) | HCl | H₂O² | H₂O | (%) | (%) | (%) | (%) | (%) | (%) | (%) | (%) |
| 1 | 1 (30 g) | 1 | 3.43 | 1.76 | — | 37.0 | | 5.9 | — | — | — | — |
| 2 | 1 (24 g) | 1.23 | 4.20 | 0 | 64 | 46.8 | 41.4 | 4.2 | 36 | 9.3 | 22.3 | <0.1 |
| 3 | 1 (20 g) | 1.50 | 5.13 | 0 | 62 | 42.8 | 41.8 | 0.61 | 38 | 14.3 | 26.4 | 0.11 |
| 4 | 1 (20 g) | 1.69 | 5.76 | 0 | 67 | 40.0 | 47.7 | 0.31 | 33 | 10.0 | 28.0 | <0.1 |

¹Reaction time was 60 min; PA was analyzed based on total weight of each phase.
²From 37% HCl.

Process II. The experiments in this Process could be considered as Process I optimization, resulting in minimized volatilization of HCl and increased product yields. Precipitation was observed when the reactor reached temperatures above 88° C. Analytical results in this Process yielded a higher PA content in the collected liquid layer than in Process I.

TABLE 8
Reactant mole ratios and result summary for reaction runs - Process II

| | Reaction | Reactant Mole Ratio¹ | | | | Liquid Phase | | | | Wet Precipitate (NH₄Cl) | | | |
|---|---|---|---|---|---|---|---|---|---|---|---|---|---|
| | time | PN | | | Added | Mass | PA | H₂O | PN | Mass | PA | H₂O | PN |
| Reaction | (min) | (mass) | HCl | H₂O² | H₂O | (%) | (%) | (%) | (%) | (%) | (%) | (%) | (%) |
| 1 | 80 | 37.6 g | 1 | 3.43 | 0 | 66.2 | 54.3 | 31.9 | 6.0 | 33.8 | 8.5 | 15.9 | 0.06 |
| 2 | 60 | 35.0 g | 1.25 | 4.29 | 0 | 65.4 | 54.8 | 39.5 | 0.53 | 34.6 | 8.5 | 17.1 | Trace |

¹Normalized to amount of PN.
²From 37% HCl.

Process III. In this process, a fixed weight (15 g) of PN was used and no water was added in all experiments. As soon as PN was added onto HCl and H₂SO₄ mixture, the reactor temperature was raised by the heat of reaction to 80° C. in less than 3 min. There was no precipitation or phase separation until the reaction temperature had reached to 88 to 90° C. When cooled to room temperature, products in Reaction 1 to 5 have three phases. The top phase is mostly PA. The middle liquid layer is a mixture of ammonium bisulfate and ammonium chloride solution. The bottom layer is ammonium chloride solid. Again, the precipitate NH₄Cl was removed by vacuum filtration. Each liquid layer was analyzed separately and the results are tabulated in Table 9.

TABLE 9
Reactant mole ratios and result summary for reaction runs in Process III. Reaction 6
has only observation.

| | Reaction | Reactant Mole Ratio | | | | Upper Phase | | | | Lower Phase | | | |
|---|---|---|---|---|---|---|---|---|---|---|---|---|---|
| | time | | | | Total | Vol | PA | H₂O | PN | Vol | PA | H₂O | PN |
| Reaction | (min) | PN¹ | HCl | H₂SO₄ | H₂O | (mL) | (%) | (%) | (%) | (mL) | (%) | (%) | (%) |
| 1 | 60 | 1 | 1 | 0.35 | 3.43 | — | 97.2 | 2.53 | Trace | — | 21.3 | — | Trace |
| 2 | 60 | 1 | 1 | 0.35 | 3.43 | 11.5 | 97.0 | 2.58 | Trace | 16.5 | 14.6 | 31.4 | Trace |
| 3 | 60 | 1 | 1 | 0.5 | 3.43 | 9.5 | 97.0 | 2.54 | Trace | 20 | 20.8 | 27.4 | Trace |
| 4 | 60 | 1 | 0.9 | 0.35 | 3.08 | 10.0 | 96.2 | 2.51 | Trace | 16 | 20.0 | 24.4 | Trace |
| 7 | 60 | 1 | 1 | 0.35 | 3.43 | Conversion of PN to total PA: 91.8% | | | | | | | |
| 8 | 90 | 1 | 0.525 | 0.525 | 3.63 | 13.0 | 76.6 | — | 9.5 | 17.9 | 10.0 | — | 2.25 |
| 9 | 90 | 1 | 0.75 | 0.525 | 3.58 | 14.5 | 96.9 | 2.86 | Trace | 17 | 8.0 | 28.4 | Trace |

¹15 g of PN was used in all reactions.

The results in Table 9 indicate the total mineral acid requirement for completion of the reaction within 90 minutes to be in excess of a 1.05 acid:PN ratio as reaction 8 resulted in incomplete conversion of PN to PA. Although inclusion of H₂SO₄ at a H₂SO₄:PN ratio of 0.35 was sufficient to generate the bi-layer liquid phase, the optimum PN:HCl:H₂SO₄:H₂O ratio is closer to the one in reaction 9 as it maximized the yield of PA retrievable from the upper layer and minimized the amount of PA in the lower layer. Further reactant ratio optimization will need to be pursued if the mixed acid approach is deemed to be of economic advantage to PN hydrolysis with H₂SO₄ alone.

For further evaluation of PA in the upper layer, FT-IR analysis was performed using sample from reaction run 1 and compared to a standard PA with 99% purity. There was a very close correspondence between the two spectra.

### Discussion

Propionitrile is effectively and rapidly hydrolyzed to PA with concentrated HCl in a monophasic reaction, with large portion of the resulting NH₄Cl precipitating upon saturation in the PA/water solution allowing for convenient recovery of a sizeable fraction of the generated ammonium salt.

Hydrolysis with gradual temperature increase is more efficient due to reduced initial HCl vaporization. The collected products have higher propionic acid concentration and less residual PN. However, the reactions of both Process I and II result in the generation of dilute PA (45-55%) with substantial amounts of soluble NH₄Cl.

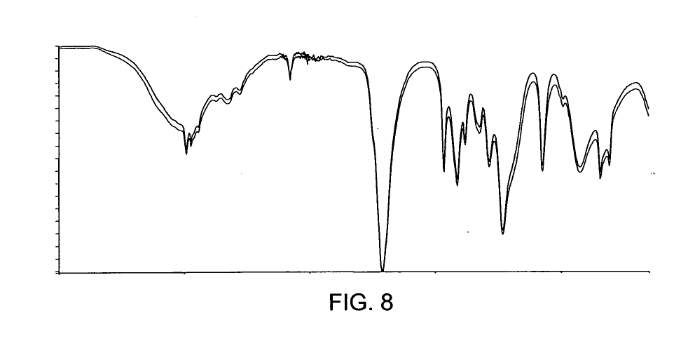

Experiments as outlined in Process III have shown a new, effective process to convert PN to PA. This combines the rapid rate of reaction obtained with HCl with the ease of PA recovery seen in H₂SO₄ experiments set out in Example 1. The reaction was near complete within 90 min and produced the distinct 2-layer liquid phase characteristic for H₂SO₄ hydrolysis of PN. Upon collection of the upper phase, 97% pure propionic acid was obtained. Analytical results found only a trace of PN remaining in both liquid layers when sufficient acid (HCl+H₂SO₄) was included in the reaction. When comparing all experimental results in this process, hydrolysis with 0.75 mole HCl and 0.525 mole H₂SO₄ per mole of PN yielded the largest upper-phase volume of high PA purity. We confirmed the equivalence of PA generated by this process to a PA standard using FTIR spectra comparison (FIG. 8).

### Conclusion

In summary, propionitrile could be hydrolyzed to propionic acid either with concentrated HCl or concentrated HCl and H₂SO₄. The hydrolysis can be achieved within a few hours and products can be separated by phase separation and filtration. When only concentrated HCl is being used, propionic acid will be produced in the form of a solution with 40% of water and soluble ammonium chloride. Extensive distillation and salt crystallization would be needed if pure propionic acid is desired from this process. Using a combination of HCl and H₂SO₄ in the reaction results in the generation of propionic acid with 97 percent purity in the upper phase of the finished product. For further purification of propionic acid in the upper layer, light distillation will remove the residual water in this phase similar to the upper phase in H₂SO₄—only reaction.

### EXAMPLE 3

### Upper and Lower Water Inclusion Limits in Sulfuric Acid Hydrolysis of Propionitrile for Easy Recovery of Propionic Acid

As described in previous examples, sulfuric acid (H₂SO₄) hydrolyzes propionitrile (PN) into propionic acid (PA) and a biphasic reaction was established. The upper phase of the final reaction mixture contained high purity PA and the lower phase was highly concentrated ammonium bisulfate (ABS). This biphasic separation should be amenable to an industrial process for the production of PA, and this requires the establishment of optimal reaction conditions to maximize reaction rate and recovery of PA, while maintaining low reactant costs.

Several optimization reactions were conducted to evaluate the effect of a range of reactant ratios on the hydrolysis of propionitrile by H₂SO₄. The first experiment evaluated the impact of water as well as sulfuric acid concentrations on reaction rate and extent using a 3×3 factorial design. The second experiment focused on the effect of varying water inclusion at a single sulfuric acid:propionitrile ratio on phase formation and propionic acid partition into the lower phase.

### Materials and Methods

All experiments employed 40 mL glass vials fitted with 8″(⅛″ID) glass tube condensers that help prevent PN vapors from escaping and allow the reactions to be run at atmospheric pressure. The first two experiments included 27 vials each, placed into a 90° C. shaking water bath. Reactant ratios varied from one vial to another, however the total mass of reactants was constant at 30 g. The propionitrile used had a labeled concentration of 99.54%, water was de-ionized and H₂SO₄ had a labeled concentration of 95.9%. Response parameters in the first experiment were PN and PA concentrations in the entire reaction volume, as well as in the individual upper and lower phases. Ammonia (i.e., ABS) was determined for the entire vial contents in the first experiment but not in the second. PN and PA were determined in the separated phases and yields were directly determined.

Gas chromatography. Fifty (50) μL of the test solution was drawn into a 100 μL Hamilton gas-tight syringe and weighed to the nearest 0.1 mg on a Mettler AE160 balance. This aliquot was transferred into sealed 2-mL GC vials containing de-ionized water. Vials were vortexed and injected into the GC and PN, PA, and PM separated using the GC conditions shown in Example 1. Working standard solutions of PN, PA and PM were prepared to cover the expected range of reactant and product concentrations for a 3×3 factorial experiment using H₂SO₄:PN mole ratios of 0.9, 1.075 and 1.25; water:PN mole ratios of 2, 3, and 5; and reaction times of 3, 7.5 and 10 hours. The standards used were propionitrile (99%, Sigma-Aldrich) and propionic acid (99%, Fisher Scientific). The working standards were prepared for analysis in the same manner as the samples.

Ammonia was determined using Nessler's reagent. In Exp. 1, 1-μL or 2-μL—aliquots of test solutions were transferred into 4 mL de-ionized water in 12×75mm glass test tubes. In Exp. 2 and 3, 250-μL aliquots of diluted samples were further diluted in 30 mL of de-ionized water in a 50 mL plastic centrifuge tube. One hundred (100) μL of this dilution was transferred into 4 mL of water in test tubes. One hundred (100) μL of Nessler's Reagent (Aqua Solutions, CY183A) was pipetted into test tubes and vortexed at 1400 rpm for 2-4 seconds. Standard solutions of ammonium sulfate (99.9%, Sigma-Aldrich, ACS reagent grade) were prepared in the same manner and the color was allowed to develop for 20-30 minutes. Tubes were read on a Beckman DU640 spectrometer at 425 nm.

Experimental Design. In Experiment 1, water:PN and H₂SO₄:PN mole ratios were varied in a 3×3 full factorial as described above and vials were pulled from the water bath at the specified three different times. The responses measured were residual PN in the reaction vial, PA produced in the reaction and ammonium bisulfate (ABS) generated. PN was expressed as the percentage of the remaining PN divided by the initial. PA and ABS are expressed as a percentage of the generated product divided by the stoichiometric quantities expected.

TABLE 10
Experiment 2: water:PN mole ratios and times tested in the experiment.
H₂SO₄:PN mole ratio was held at 1.05.

| | Water:PN | Time |
|---|---|---|
| Vial | (mole ratio) | (Hours) |
| 1 a, b, c | 2.25 | 2, 6, 22 |
| 2 a, b, c | 2.5 | 2, 6, 22 |
| 3 a, b, c | 3.0 | 2, 6, 22 |
| 4 a, b, c | 3.5 | 2, 6, 22 |
| 5 a, b, c | 4.0 | 2, 6, 22 |
| 6 a, b, c | 4.5 | 2, 6, 22 |
| 7 a, b, c | 5.0 | 2, 6, 22 |
| 8 a, b, c | 7.5 | 2, 6, 22 |
| 9 a, b, c | 10.0 | 2, 6, 22 |

In Experiment 2, H₂SO₄:PN was held constant at 1.05 and the amount of water was varied from a near stoichiometric water:PN mole ratio of 2 to an excess of 10 (see Table 10). Responses measured were the levels of PN remaining and PA generated after 2, 6, and 22 h. As seen in previous work distinct phases are generated, and in this experiment levels of PA and PN were measured in each phase and the yields recovered were calculated based on measured levels of PA in the phases compared to initial reactant concentrations.

### Results & Discussion

Experiment 1. Observations made after reaction for 3, 7.5 and 13 h reveal a wide variation in the formation of phases as well as degree of crystallization. At high sulfuric levels and low water inclusion no phase separation is generated. At low water inclusion crystallization occurred whereas with higher water content the lower phase is uncrystallized and the phase separation is distinct. The other vials are intermediate in the degree of crystallization and the clarity of the generated upper phase.

The initial weight of reactants was compared to the final weight. With a water:PN ratio of 2, significant weight losses were observed, up to 7.9% (Table 11). These losses were due to large vapor releases from the reflux condensers. The releases in some cases were a white smoke-like vapor, which is likely fuming sulfuric acid (H₂S₂O₇), also called oleum. Additionally, the higher H₂SO₄ concentrations appear to discolor the resulting reaction products to light amber.

TABLE 11
Reaction analysis of Experiment 2, including GC and ammonia assay results (%,
wt/wt) on the entire vial contents and estimated phase volumes (%, vol/vol)

| | | | Upper | Lower | | | | | |
|---|---|---|---|---|---|---|---|---|---|
| | | | Phase | Phase | | | | | |
| | SA:PN | Water:PN | Vol | Vol.(% | Mass | | | | |
| Time | (mole | (mole | (% of | of | Loss | | PA | PM | ABS |
| (h) | ratio) | ratio) | total) | total) | (%) | PN (%) | (%) | (%) | (%) |
| 3 | 0.9 | 2 | 2 | 98 | 1.8 | 1.4 | 37.2 | 6.5 | 36.4 |
| 3 | 0.9 | 3 | 48 | 52 | 0.2 | 6.3 | 32.2 | 0.8 | 42.8 |
| 3 | 0.9 | 5 | 30 | 70 | 0.4 | 12.1 | 19.3 | 0.1 | 20.2 |
| 3 | 1.07 | 2 | 2 | 98 | 1.7 | 0.6 | 28.1 | 3.1 | 42.7 |
| 3 | 1.07 | 3 | 36 | 64 | 0.2 | 1.6 | 39.1 | 1.6 | 45.3 |
| 3 | 1.07 | 5 | 30 | 70 | 0.4 | 7.3 | 27.2 | 0.1 | 28.7 |
| 3 | 1.25 | 2 | 0 | 100 | 3.6 | 0.5 | 25.3 | 2 | 30.2 |
| 3 | 1.25 | 3 | 20 | 80 | 0.2 | 0.4 | 39.9 | 1.8 | 45.7 |
| 3 | 1.25 | 5 | 29 | 71 | 0.7 | 3.2 | 28.8 | 0.1 | 34.1 |
| 7.5 | 0.9 | 2 | 14 | 86 | 1.2 | 0.8 | 39.7 | 5.3 | 46.1 |
| 7.5 | 0.9 | 3 | 46 | 54 | 0.2 | 4.8 | 34.4 | 0.2 | 43.2 |
| 7.5 | 0.9 | 5 | 34 | 66 | 0.5 | 8.7 | 23.1 | 0 | 33.6 |
| 7.5 | 1.07 | 2 | 5 | 95 | 3 | 0.5 | 36.1 | 1.8 | 39.1 |
| 7.5 | 1.07 | 3 | 45 | 55 | 0.3 | 1.1 | 36.6 | 0.1 | 48.5 |
| 7.5 | 1.07 | 5 | 33 | 67 | 0.5 | 4.1 | 25.7 | 0 | 31.5 |
| 7.5 | 1.25 | 2 | 0 | 100 | 3.4 | 0.5 | 35.6 | 1 | 27.6 |
| 7.5 | 1.25 | 3 | 40 | 60 | 0.2 | 0.3 | 38.1 | 0.1 | 46 |
| 7.5 | 1.25 | 5 | 31 | 69 | 1 | 1.6 | 29.5 | 0 | 36.2 |
| 13 | 0.9 | 2 | 30 | 70 | 0.6 | 0.9 | 42 | 2.4 | 52.7 |
| 13 | 0.9 | 3 | 47 | 53 | 0.4 | 4.2 | 37 | 0 | 40.2 |
| 13 | 0.9 | 5 | 36 | 64 | 0.5 | 6.8 | 28.1 | 0 | 33.1 |
| 13 | 1.07 | 2 | 0 | 100 | 3.9 | 0.4 | 38.2 | 1.1 | 38.3 |
| 13 | 1.07 | 3 | 43 | 57 | 0.3 | 0.8 | 40.8 | 0 | 50.6 |
| 13 | 1.07 | 5 | 33 | 67 | 0.5 | 2.9 | 29.7 | 0 | 35.2 |
| 13 | 1.25 | 2 | 0 | 100 | 7.9 | 0.4 | 36.6 | 0.7 | 30.7 |
| 13 | 1.25 | 3 | 41 | 59 | 0.3 | 0.3 | 40.5 | 0 | 44.4 |
| 13 | 1.25 | 5 | 33 | 67 | 0.4 | 1 | 31.8 | 0 | 38.8 |

The optimum reactant ratios were established via surface response analysis of ABS data using Statgraphics Plus V5.1 statistical analysis software. (Manugistics, Inc., Rockville, Md.). The concentration of total ammonium ions released from the nitrile group during hydrolys is a direct measure of the conversion of propionitrile into propionic acid. Specifically, ammonium bisulfate is formed after hydrolysis of propionamide, the intermediate product of propionitrile hydrolysis. For every mole of PN converted into PA, one mole of ABS is generated. Because propionamide is not detected using the ammonia assay method, this is an appropriate assay to measure rate of PN to PA conversion.

FIG. 6 is a surface response plot of the molar conversion to ABS as influenced by H₂SO₄ and water content in the bounds of the experiment. Each plot shows the molar yield expected at the plotted conditions. Molar yield is calculated from the total moles of captured ABS divided by the initial moles of PN in the reaction vial. For example, from FIG. 6A one can read that the expected reaction conversion of PN to ABS is 90% by 3 h when the concentrations of H₂SO₄ and water are 1.2 and 3.75, respectively.

Low yields of ABS are seen at the low and high water inclusion. Suboptimal yields at the low water:PN ratio of 2 are primarily due to the vapor losses. Suboptimal yield at low H₂SO₄ inclusion is due to this reactant being the limiting reagent rather than PN. It is also evident that H₂SO₄ content and water inclusion affect the rate of reaction. Based on surface response analysis of this experiment, the optimum reactant ratios would be 1.22 moles sulfuric and 4.02 moles of water with a reaction time of 11.6 h. The rate of reaction will be greater with H₂SO₄ in excess, however at higher H₂SO₄ concentrations discoloration of products, production of excess vapor, and crystallization of the lower phase can occur.

Experiment 2. The second experiment continued to pinpoint the optimum water:PN ratio. In this experiment the effect of a wider range of water content was explored with H₂SO₄:PN held at a mole ratio of 1.05. In this experiment analysis was done by GC. The phases generated were sampled by pulling an aliquot from the actual liquid phase. In reactions run with insufficient water it was not possible to sample the lower phase in this manner due to the crystallization. For every sample vial, the lower phase was also physically isolated and weighed and assayed by GC in order to get a measure of the concentration of products in each phase.

TABLE 12
Reaction analysis of Experiment 2, including the GC results of upper and lower
phases, estimated phase volumes and measured mass loss; lower phase concentrations
are the result of the average of the two assay methods (wt/wt).

| | | Upper Phase | | Reaction | Upper | Upper | Average | Average |
|---|---|---|---|---|---|---|---|---|
| Water:PN | | Volume | Lower | Mass | Phase | Phase | Lower | Lower |
| (mole | Time | (% of | Phase Volume | Loss | PN | PA | Phase | Phase |
| ratio) | (h) | total) | (% of total) | (%) | (%) | (%) | PN (%) | PA (%) |
| 2.25* | 2 | 0 | 100 | 15.0 | 0.0 | 0.0 | 0.0 | 28.7 |
| 2.5 | 2 | 0 | 100 | 0.1 | 0.0 | 0.0 | 1.0 | 30.3 |
| 3.0 | 2 | 47 | 53 | 0.2 | 6.1 | 59.5 | 1.7 | 23.7 |
| 3.5 | 2 | 35 | 65 | 0.6 | 10.8 | 77.8 | 1.1 | 10.0 |
| 4.0 | 2 | 33 | 67 | 0.4 | 16.8 | 64.5 | 1.4 | 10.2 |
| 4.5 | 2 | 29 | 71 | 0.4 | 20.7 | 53.5 | 2.7 | 9.9 |
| 5.0 | 2 | 25 | 75 | 0.6 | 28.0 | 53.3 | 3.2 | 9.3 |
| 7.5* | 2 | 16 | 84 | 13.7 | 65.9 | 20.6 | 6.5 | 5.6 |
| 10.0 | 2 | 15 | 85 | 0.4 | 79.0 | 8.2 | 5.8 | 3.2 |
| 2.25 | 6 | 12 | 88 | 0.6 | 0.0 | 94.4 | 0.1 | 30.1 |
| 2.5 | 6 | 35 | 65 | 0.2 | 0.5 | 105.5 | 0.1 | 16.3 |
| 3.0 | 6 | 46 | 54 | 0.3 | 3.5 | 99.1 | 0.2 | 6.4 |
| 3.5 | 6 | 41 | 59 | 1.6 | 6.4 | 100.9 | 0.2 | 3.4 |
| 4.0 | 6 | 40 | 60 | 0.4 | 10.6 | 88.9 | 0.5 | 4.3 |
| 4.5 | 6 | 36 | 64 | −1.6 | 10.9 | 77.2 | 0.7 | 5.8 |
| 5.0 | 6 | 34 | 66 | 0.5 | 15.2 | 72.9 | 1.2 | 6.2 |
| 7.5 | 6 | 20 | 80 | 0.5 | 34.2 | 46.6 | 3.5 | 7.8 |
| 10.0 | 6 | 15 | 85 | 0.4 | 58.0 | 26.1 | 5.3 | 6.0 |
| 2.25 | 22 | 47 | 53 | 0.5 | 0.0 | 98.3 | 0.2 | 6.0 |
| 2.5 | 22 | 45 | 55 | 0.1 | 0.1 | 100.7 | 0.1 | 5.6 |
| 3.0 | 22 | 45 | 55 | 0.4 | 1.4 | 95.1 | 0.1 | 5.6 |
| 3.5 | 22 | 42 | 58 | 0.4 | 2.9 | 86.5 | 0.1 | 2.7 |
| 4.0 | 22 | 41 | 59 | 0.6 | 3.3 | 96.6 | 0.2 | 4.1 |
| 4.5 | 22 | 38 | 62 | 0.5 | 4.7 | 80.7 | 0.2 | 4.2 |
| 5.0* | 22 | 32 | 68 | 8.0 | 1.4 | 99.8 | 0.2 | 5.2 |
| 7.5 | 22 | 25 | 75 | 0.4 | 9.1 | 63.7 | 0.9 | 8.7 |
| 10.0 | 22 | 16 | 84 | 0.7 | 17.8 | 57.7 | 1.4 | 8.8 |

*Reaction vials excluded from analysis surface response due to excessive sample loss

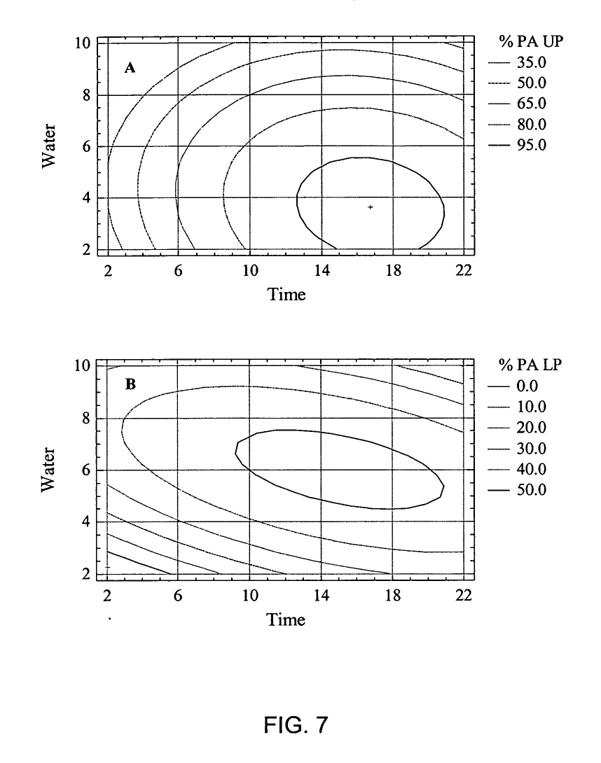

FIGS. 7A-B include 2-D contour plots of the surface response for maximum PA recovery in the upper phase and residual PA recovered from the lower phase. Based on this analysis the optimum reaction conditions for maximum recovery of PA from the upper phase and minimum loss in the lower phase would be in the region of overlap of the >95% PA in the upper phase and <0.0% PA in the lower phase. The optimum response for PA in the upper phase is achieved with a water:PN ratio of 3.6 after 15.8 h and the minimum PA in the lower phase is obtained with a water:PN ratio of 6.0 after 14.1 h. If a line is drawn between the maximum and minimum points and through the intersection of the 95 and 0% contours the estimated optimum reactant ratio would be 4.5 with a reaction time of 16 h.

Using the conditions of Experiment 2 as constraints for the surface response in Experiment 1, the surface model predictions can be compared. With 1.05 moles of H₂SO₄ and a 16-h reaction time, the surface response model in Experiment 1 predicts a maximum ABS yield of 86.7% with water:PN ratio of 3.0, which is near the predicted Experiment 2 optimum water:PN ratio of 3.6.

### Discussion

The goal of these experiments was to evaluate how reactant concentrations, especially water, affect rate and yield of the hydrolysis of propionitrile with H₂SO₄. From Experiment 1 it is clear that increased H₂SO₄ concentrations will accelerate the hydrolysis of PN into PA, whereas an optimum water:PN ratio above the stoichoimetric ratio of 2 is likely. The second experiment confirmed the first in that increased water content above a water:PN ratio of 4 also slowed the overall reaction. In other words, a higher ratio of acid to nitrile increases reaction rate, although this is constrained at higher acid to PN ratio by lower yields due to vapor loss from vials. Increasing H₂SO₄ beyond 1.25 moles per mole of PN with less than 3 moles of water is not preferred due to crystallization of the lower phase and the potential to discolor the final product as seen in FIG. 1. Hydration of H₂SO₄ is highly exothermic. The reaction forms hydronium ions, as such: H₂SO₄+H₂O→H₃O⁺+HSO₄⁻. H₂SO₄ hydration is thermodynamically favorable and H₂SO₄ is, therefore, an excellent dehydrating agent as seen in the reactions with low water inclusion.

The two-phase phenomenom warrants further discussion. First, as the reaction proceeds, the reactant/product mix separates into two distinct liquid phases, one organic and one aqueous. Anhydrous H₂SO₄ is a very polar liquid that dissociates itself by autoprotolysis: 2 H₂SO₄−>H₃SO₄⁺+HSO₄⁻. This allows protons to be highly mobile in H₂SO₄, and makes H₂SO₄ an excellent solvent for many reactions. In fact, the equilibrium is more complex than shown above. 100% H₂SO₄ contains the following species at equilibrium (figures shown as mmol per kg solvent): HSO₄⁻(15.0), H₃SO₄⁺(11.3), H₃O⁺(8.0), HS₂O₇(3.6), H₂O (0.1). The high polarity of H₂SO₄ and the stabilization of that polarity by the formation of additional HSO₄³¹ is likely to contribute to the separation of an organic, apolar layer from the aqueous layer in the reaction of H₂SO₄ with propionitrile. Once the reaction is complete, the organic, apolar phase consists almost entirely of apolar propionic acid, which is 99.99% protonated at pH 1 given its pKa of 4.87, and the aqueous phase is almost devoid of propionic acid unless the concentration of water in the aqueous phase is high enough to reduce the polarity. From the second experiment, it appears that recovery of PA from the upper phase is constrained by crystallization of the reaction mix at lower water content and is also reduced at higher water levels by slower reaction and partition of PA into the lower phase. At higher water levels the solubility of PA is increased in the lower phase and recovery of PA from the upper phase would be reduced. At lower water inclusion it seems likely that crystallization of the lower phase may tend to trap PA in the lower phase, again resulting in lowered recovery from the upper phase. The results from both experiments contribute to the definition of the minimum required amount of water to maintain a liquid reaction environment (water:sulfuric acid to be at least 2.5) and the maximum inclusion rate of water to maximize reaction rate and maintain substantially pure organic and aqueous phases (water:PN to be at most 6). The optimum water:PN ratio is between 3 and 4, but may need slight upward adjustment in case of a sulfuric acid: PN ratio in excess of 1.25.

### Conclusion

Several sulfuric acid hydrolysis optimization reactions were conducted to establish optimum reactant ratios. Reaction rate increases linearly with increasing H₂SO₄:PN ratio, whereas maximum reaction rate and phase purity require an optimum water:PN ratio between 3.5 and 4.0 depending on H₂SO₄ level. In a subsequent experiment the water:PN ratio optimum was confirmed to be 3.6 based on maximum yield of propionic acid in the upper phase. Increasing the water:PN ratio above 6 significantly increases the amount of PA remaining in the lower phase.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A method of simultaneously producing from a selected nitrile the corresponding carboxylic acid and ammonium salt; comprising the steps of:
(a) combining in a reaction vessel near stoichiometric amounts of the nitrile and a strong acid, as well as water;
(b) forming a reaction mixture comprising an upper organic phase and a lower aqueous phase; and
(c) heating the reaction mixture to above about 50° C., and preferably above about 85° C. to produce first the corresponding amide which migrates to the lower phase followed by the carboxylic acid which is present primarily in the upper phase and the ammonium salts are present primarily in the lower phase, and wherein the carboxylic acid comprises less than about 15% by volume of the lower phase.

2. A method as defined in claim 1, further comprising the step of removing at least part of the upper phase and recovering the carboxylic acid.

3. A method as defined in claim 2, wherein the step of recovering the carboxylic acid comprises distillation.

4. A method as defined in claim 3, further comprising the step of returning the distillate to the reaction mixture.

5. A method as defined in claim 1, further comprising the step of removing at least part of the lower phase.

6. A method as defined in claim 5, further comprising the step of adding ammonia to the removed lower phase to neutralize pH of the lower phase.

7. A method as defined in claim 1, wherein the reaction mixture is heated, assuming atmospheric pressure, to between about 50° C. and about 100° C., and preferably between about 85° C. and about 95° C.

8. A method as defined in claim 1, wherein the strong acid consists of sulfuric acid.

9. A method as defined in claim 1, wherein the strong acids consist of a combination of sulfuric acid and hydrochloric acid.

10. A method as defined in claim 1, wherein the nitrile is an aliphatic nitrile.

11. A method of simultaneously producing from propionitrile propionic acid and ammonium bisulfate; comprising the steps of:
(a) combining in a reaction vessel, on a mole basis, one part propionitrile, between about one and one and one-half parts sulfuric acid, and between about two and one-half and ten parts water;
(b) forming a reaction mixture comprising an upper organic phase and a lower aqueous phase; and
(c) heating the reaction mixture to above about 50° C. and, assuming atmospheric pressure, preferably between about 80° C. and about 95° C. to produce first propinamide which migrates to the lower phase followed by propionic acid which is present primarily in the upper phase and ammonium bisulfate which is present primarily in the lower phase and wherein propionic acid comprises less than about 15% by volume of the lower phase.

12. A method as defined in claim 11, wherein the mole ratio of sulfuric acid to propionitrile is between 1 and 1.25.

13. A method as defined in claim 11, wherein the mole ratio of water to propionitrile is between 2.5 and 6.

14. A method as defined in claim 11, wherein a combination of hydrochloric and sulfuric acids are used in place of sulfuric acid, and wherein the mole ratio of hydrochloric acid to sulfuric acid is between about 0.1: 1 and about 1:0.3.

15. A method of continuously simultaneously producing from a selected aliphatic nitrile the corresponding carboxylic acid and ammonium salts; comprising the steps of:
(a) continuously adding to a first reaction vessel near stoichiometric amounts of the nitrile, a strong acid, and water to form a reaction mixture;
(b) heating the reaction mixture to above about 50° C., and preferably above about 85° C.;
(c) continuously transferring to a second reaction vessel the reaction mixture;
(d) adjusting the in-flow and out-flow of the first reaction vessel to provide an average residence time of the reaction mixture in the first reaction vessel sufficient to allow the reaction to proceed to at least 50% completion;
(e) forming in the second reaction vessel a reaction mixture comprising an upper organic phase and a lower aqueous phase at a temperature above about 50° C., and preferably above about 85° C. to produce the carboxylic acid which is present primarily in the upper phase and the ammonium salts are present primarily in the lower phase, and wherein the carboxylic acid comprises less than about 15% by volume of the lower phase; and
(f) continuously removing from the second reaction vessel the upper phase and separately the lower phase.
